(19) United States
(12) Patent Application Publication
(10) Pub. No.: US 2011/0178541 A1
(43) Pub. Date: Jul. 21, 2011

(54) VIRTUAL ULTRASONIC SCISSORS

(75) Inventor: Haim Azhari, Doar-Na Misgav (IL)

(73) Assignee: SLENDER MEDICAL, LTD., Herziya (IL)

(21) Appl. No.: 13/061,269

(22) PCT Filed: Sep. 13, 2009
(86) PCT No.: PCT/IL2009/000893

§ 371 Date: Apr. 6, 2011

Related U.S. Application Data
(60) Provisional application No. 61/096,516, filed on Sep. 12, 2008.

(51) Int. Cl. A61B 17/32

(52) U.S. Cl. 606/167

(57) ABSTRACT

Apparatus is provided, which includes at least first and second focused ultrasonic transducers, which are arranged facing each other, and a controllable energy source. The energy source is configured to activate the focused ultrasonic transducers to simultaneously generate respective first and second focused ultrasound beams having respective first and second focal zones located in close proximity to each other. Other embodiments are also described.

### CROSS-REFERENCE TO RELATED APPLICATIONS

The present patent application claims the benefit of U.S. Provisional Application 61/096,516, filed Sep. 12, 2008, entitled, “Virtual Ultrasonic Scissors—A non-invasive method for tissue treatment,” which is incorporated herein by reference.

This application is related to U.S. Provisional Patent Application 61/096,419, entitled, “A device for ultrasound treatment and monitoring tissue treatment,” filed Sep. 12, 2008, and to an international patent application filed on even date herewith which claims the benefit of the '419 application, and is entitled, “A device for ultrasound treatment and monitoring tissue treatment.” Both of these applications are incorporated herein by reference.

### FIELD OF THE INVENTION

Some embodiments of the present invention relate generally to cosmetic and therapeutic ultrasound. More particularly, some embodiments of the present invention relate to techniques for non-invasively destroying selected tissue regions for cosmetic or therapeutic purposes.

### BACKGROUND OF THE INVENTION

Non-invasive tissue treatment by high intensity focused ultrasound (HIFU) has become commercially available in recent years. Systems are commercially available for therapeutic procedures (e.g., treatment of tumors of the uterus, breast, and prostate), and for cosmetic procedures (e.g., lipolysis and body contouring). In general, the currently available tissue treatment techniques destroy tissue either by thermal ablation, or by ultrasonically-induced cavitation.

Tissue treatment by thermal ablation is associated with substantial temperature elevation. Such treatment thus utilizes thermal monitoring, which is generally difficult to implement non-invasively without MRI. In contrast, ultrasonically-induced cavitation is a “cold” tissue treatment technique. However, the cavitation process is difficult to control and monitor, because the generated bubbles may be carried away from the treated area by the blood circulation.

PCT Publication WO 07/102,161 to Azhari et al., which is incorporated herein by reference, describes apparatus for lipolysis and body contouring of a subject. The apparatus includes a housing adapted for placement on tissue of the subject. The apparatus also includes a plurality of acoustic elements disposed at respective locations with respect to the housing, including at least a first and a second subset of the acoustic elements, wherein the first subset is configured to transmit energy in a plane defined by the housing, such that at least a portion of the transmitted energy reaches the second subset. Other embodiments are also described.

PCT Publication WO 06/018837 to Azhari et al., which is incorporated herein by reference, describes a method of damaging a target tissue of a subject. The method is described as comprising: (a) imaging a region containing the target tissue; (b) determining a focal region of a damaging radiation; (c) positioning the focal region onto the target tissue; and (d) damaging the target tissue by an effective amount of the damaging radiation. The determination of the focal region is described as being performed by delivering to the region bursts of ultrasonic radiation from a plurality of directions and at a plurality of different frequencies, and passively scanning the region so as to receive from the region ultrasonic radiation having at least one frequency other than the plurality of different frequencies.

PCT Publication WO 01/92846 to Azhari et al. describes a system for the localization of target objects using acoustic signals. The system comprises an acoustic transducer; acoustic reflecting means; processing means and output means. The transducer is adapted to transmit acoustic signals to a target object, receive superposed echoes from the target object; directly from the target object and indirectly, reflected by said acoustic reflecting means, and transmit an electrical signal corresponding to the received superposed acoustic signal to said processing means. The processing means is adapted to compute the position of the target object and output the position through said output means.

The following references may be of interest:
U.S. Pat. No. 4,355,643 to Laughlin et al.
U.S. Pat. No. 5,143,063
U.S. Pat. No. 5,573,497 to Chapelon
U.S. Pat. No. 5,575,772 to Lennox
U.S. Pat. No. 5,601,526 to Chapelon et al.
U.S. Pat. No. 5,665,053 to Jacobs
U.S. Pat. No. 5,743,863 to Chapelon
U.S. Pat. No. 6,113,558 to Rosenschein et al.
U.S. Pat. No. 6,350,245 to Cimino
U.S. Pat. No. 6,438,424 to Knowlton
U.S. Pat. No. 6,450,979
U.S. Pat. No. 6,500,141 to Ilion et al.
U.S. Pat. No. 6,508,813 to Altshuler
U.S. Pat. No. 6,607,498
U.S. Pat. No. 6,626,854
U.S. Pat. No. 6,645,162 to Friedman et al.
U.S. Pat. No. 6,730,034
U.S. Pat. No. 6,758,845 to Weckwerth et al.
U.S. Pat. No. 6,971,994
U.S. Pat. No. 7,258,674 to Cribbs et al.
US Patent Application Publication 2002/0193831
US Patent Application Publication 2003/0083536
US Patent Application Publication 2004/0039312 to Hillstead et al.
US Patent Application Publication 2004/0215110
US Patent Application Publication 2004/0217675
US Patent Application Publication 2005/0049543 to Anderson et al.
US Patent Application Publication 2005/0154295
US Patent Application Publication 2005/0154308
US Patent Application Publication 2005/0154309
US Patent Application Publication 2005/0154313
US Patent Application Publication 2005/0154314
US Patent Application Publication 2005/0154431
US Patent Application Publication 2005/0187463
US Patent Application Publication 2005/0187495
US Patent Application Publication 2005/0193451
US Patent Application Publication 2005/0261584
US Patent Application Publication 2006/0036300
US Patent Application Publication 2006/0058707 to Barthe et al.
US Patent Application Publication 2006/0094988
US Patent Application Publication 2006/0122509
PCT Publication WO 00/053263
PCT Publication WO 01/92846 to Azhari et al.
PCT Publication WO 05/065371 to Quistgaard et al.
PCT Publication WO 05/065409 to Quistgaard et al.
PCT Publication WO 05/074365
PCT Publication WO 06/080012 to Kreindel
PCT Publication WO 06/080012 to Kreindel
Akashi N et al., “Acoustic properties of selected bovine tissue in the frequency range 20-200 MHz,” J Acoust Soc Am. 98(6):3.035-9 (1995)
Laubach H J et al., “Intense focused ultrasound: evaluation of a new treatment modality for precise microcoagulation within the skin,” Dermatol Surg 34:727-734 (2008)
Moran C M et al., “Ultrasonic propagation properties of excised human skin,” Ultrasound Med Biol. 21(9):1177-90 (1995)

### SUMMARY OF THE INVENTION

In some embodiments of the present invention, apparatus and methods are provided for non-invasively destroying a target tissue region. At least one pair of focused ultrasonic transducers are arranged to induce mechanical shear forces within the tissue region. The transducers simultaneously generate ultrasound beams in close proximity to each other in the tissue region, but in opposite directions and/or with oppositely-signed pressures. The resulting shear forces tear and/or otherwise destroy at least a portion of the target tissue. The ultrasound beams can thus be considered to function as virtual scissors. Each of the transducers generates high intensity focused ultrasound (HIFU) beams, and comprises either a phased array of transducer elements, and/or one or more focused transducer elements.

This tissue destruction process is generally non-thermal, and thus avoids the need for thermal monitoring during the process. In addition, the process is highly controllable, by accurately spatially orienting the beams.

The tissue destruction process may be used for performing therapeutic procedures, typically without the need to cut the skin. Such procedures include, but are not limited to, treatment of tumors, such as of the uterus, breast, or prostate, or of varicose veins.

The tissue destruction process may also be used for performing cosmetic procedures (e.g., lipolysis (destruction of adipose tissue) or body contouring).

There is therefore provided, in accordance with an embodiment of the present invention, apparatus including:

at least first and second focused ultrasonic transducers, which are arranged facing each other; and

a controllable energy source, which is configured to activate the focused ultrasonic transducers to simultaneously generate respective first and second focused ultrasound beams having respective first and second focal zones located in close proximity to each other.

For some applications, the apparatus is configured to generate the respective beams such that a distance between respective centers of the respective focal zones is between 25% and 200% of the sum of a greatest diameter of the first focal zone and a greatest diameter of the second focal zone. For example, the beams may have respective wavelengths of between 0.15 and 1.5 mm. For some applications, the apparatus is configured to generate the respective beams such that a distance between respective centers of the respective focal zones is between 25% and 200% of the sum of a greatest diameter of the first focal zone and a greatest diameter of the second focal zone, for a time period between 0.2 and 60 seconds.

Typically, the apparatus is configured to generate the respective beams having respective opposing acoustic forces such that the beams together generate mechanical shear forces between the focal zones. In addition, the apparatus is typically configured to generate the respective beams such that the beams tear a material disposed between the focal zones.

For some applications, the apparatus is configured to generate the respective beams such that the beams do not increase a temperature of a material, having a specific heat of 4.18 J/(g*K), disposed between the focal zones, by more than 20° C. Typically, the apparatus is configured to generate the respective beams such that the beams do not cause substantial cavitation in the material.

For some applications, the apparatus further includes a support structure, to which the focused ultrasonic transducers are coupled.

For some applications, the apparatus is configured to generate the respective beams such that the beams have parallel respective axes. For some applications, the apparatus is configured to generate the respective beams such that a distance between the respective axes is between 25% and 200% of the sum of a greatest diameter of the first focal zone and a greatest diameter of the second focal zone.

For some applications, the focused ultrasonic transducers include respective single elements that are configured to generate the respective focused ultrasound beams having respective fixed focal zones. Alternatively, the focused ultrasonic transducers include respective phased arrays, and the energy source is configured to activate the arrays to generate the respective focused ultrasound beams.

For some applications, the energy source is configured to activate each of the arrays to steer its respective focused ultrasound beam in a plurality of directions during respective time periods.

For some applications, the energy source is configured to mechanically steer the respective focused ultrasound beams.

For some applications, the focused ultrasonic transducers together include a phased array arranged in a ring, and the energy source is configured to activate a first subgroup of the elements to generate the first focused ultrasound beam, and a second subgroup of the elements, different from the first subgroup, to generate the second focused ultrasound beam.

For some applications, the focused ultrasound beams are shock waves, and the focused ultrasonic transducers are configured to simultaneously generate the shock waves having the respective first and second focal zones in close proximity to each other.

For some applications, the apparatus is configured to perform a calibration procedure, in which the apparatus initially generates the first and second ultrasound beams such that the respective focal zones coincide, and thereafter adjusts a location of at least one of the focal zones such that the focal zones are in close proximity to each other, rather than coincide.

There is further provided, in accordance with an embodiment of the present invention, a method including:

identifying a target tissue region in a body of a subject; and

destroying at least a portion of the target tissue region by simultaneously generating, in opposing directions, at least first and second focused ultrasound beams having respective first and second focal zones in close proximity to each other within the target tissue region.

There is still further provided, in accordance with an embodiment of the present invention, apparatus including:

at least first and second focused ultrasonic transducers, which are arranged facing in generally a same direction; and

a controllable energy source, which is configured to activate the focused ultrasonic transducers to simultaneously generate respective first and second focused ultrasound beams having respective first and second focal zones, which are located in close proximity to each other, and have oppositely-signed pressures.

For some applications, the apparatus is configured to generate the respective beams such that a distance between respective centers of the respective focal zones is between 25% and 200% of the sum of a greatest diameter of the first focal zone and a greatest diameter of the second focal zone. For example, the beams may have respective wavelengths of between 0.15 and 1.5 mm. For some applications, the apparatus is configured to generate the respective beams such that a distance between respective centers of the respective focal zones is between 25% and 200% of the sum of a greatest diameter of the first focal zone and a greatest diameter of the second focal zone, for a time period between 0.2 and 60 seconds

Typically, the apparatus is configured to generate the respective beams having respective opposing acoustic forces such that the beams together generate mechanical shear forces between the focal zones. In addition, the apparatus is typically configured to generate the respective beams such that the beams tear a material disposed between the focal zones.

For some applications, the focused ultrasound beams are shock waves, and the focused ultrasonic transducers are configured to simultaneously generate the shock waves having the respective first and second focal zones in close proximity to each other.

For some applications, the energy source is configured to activate the focused ultrasonic transducers to generate arbitrary waveforms.

For some applications, the apparatus is configured to perform a calibration procedure, in which the apparatus initially generates the first and second ultrasound beams such that the respective focal zones coincide, and thereafter adjusts a location of at least one of the focal zones such that the focal zones are in close proximity to each other, rather than coincide.

There is additionally provided, in accordance with an embodiment of the present invention, a method including:

identifying a target tissue region in a body of a subject; and

destroying at least a portion of the target tissue region by simultaneously generating, in non-opposing directions, at least first and second focused ultrasound beams having respective first and second focal zones, which are in close proximity to each other within the target tissue region, and which have oppositely-signed pressures.

The present invention will be more fully understood from the following detailed description of embodiments thereof taken together with the drawings, in which:

### BRIEF DESCRIPTION OF THE DRAWINGS

FIGS. 1A and 1B are schematic illustrations of an ultrasonic tissue destruction system, in accordance with an embodiment of the present invention;

FIG. 2 is a schematic illustration of a configuration of ultrasonic transducers of the system of FIGS. 1A and 1B, in accordance with an application of the present invention;

FIG. 3 is a schematic illustration of another configuration of the ultrasonic transducers of the system of FIGS. 1A and 1B, in accordance with an application of the present invention;

FIG. 4 is a schematic illustration of yet another configuration of the ultrasonic transducers of the system of FIGS. 1A and 1B, in accordance with an application of the present invention;

FIG. 5 is a schematic illustration of still another configuration of the ultrasonic transducers of the system of FIGS. 1A and 1B, in accordance with an application of the present invention;

FIG. 6 is a schematic illustration of a configuration of a phased array, in accordance with an application of the present invention; and

FIG. 7 is a schematic illustration of a unidirectional transmission configuration of the tissue destruction system of FIGS. 1A and 1B, in accordance with an application of the present invention.

### DETAILED DESCRIPTION OF EMBODIMENTS

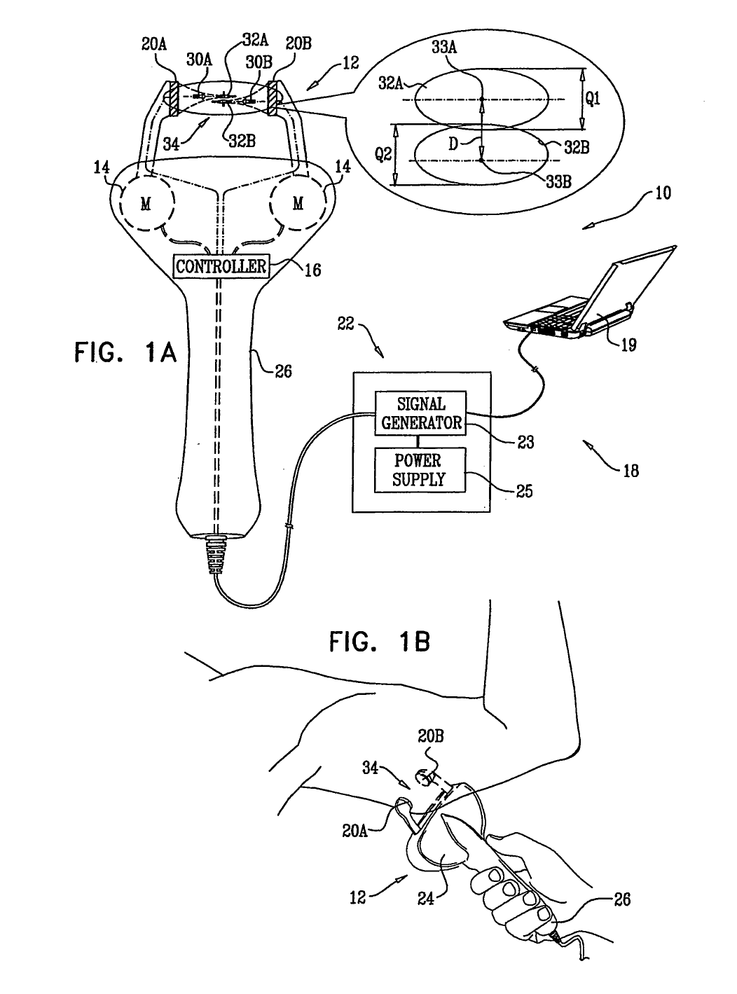

FIGS. 1A and 1B are schematic illustrations of an ultrasonic tissue destruction system 10, in accordance with an embodiment of the present invention. Tissue destruction system 10 comprises at least one pair of focused ultrasonic transducers 20A and 20B, which are arranged generally facing each other. System 10 further comprises a controllable energy source 22, which comprises a signal generator 23 and a power supply 25. Energy source 22 is configured to generate signals for activating the ultrasonic transducers.

Typically, system 10 comprises a hand-held applicator 12, to which transducers 20A and 20B are coupled. Hand-held applicator 12 comprises a housing 24 that contains and/or is coupled to components of the system, and serves as a support structure for appropriately positioning and orienting the transducers. For some applications, a portion of housing 24 is shaped so as to define a handle 26. For some applications, the hand-held applicator comprises an electromechanical system for moving the transducers, such as towards/away from each other, further/closer to housing 24, and/or otherwise with respect to each other. The electromechanical system typically comprises motors 14 for moving the transducers, an electronic controller 16, and mechanical elements.

Typically, controllable energy source 22 is located in a control unit 18 separate from applicator 12. For some applications, control unit 18 further includes a workstation 19 (such as a personal computer) for recording and analyzing signals applied by system 10, and/or activating and/or configuring energy source 22. Alternatively, one or more components of energy source 22 are contained within housing 24.

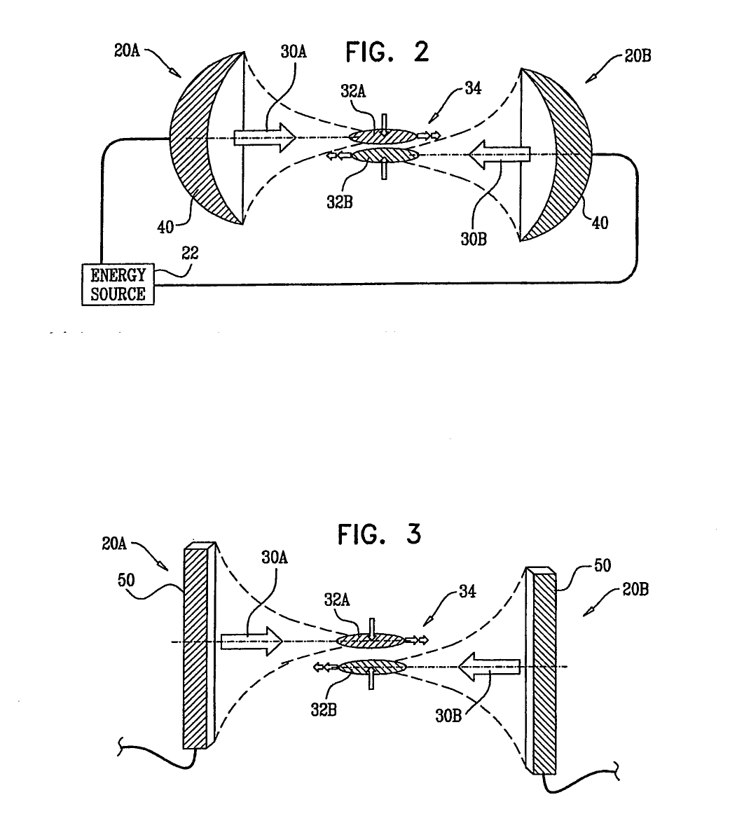

In the configuration described herein with reference to FIGS. 1A-6, transducers 20A and 20B simultaneously generate ultrasound beams 30A and 30B in parallel but opposing directions. System 10 configures the beams such that respective focal zones 32A and 32B of the beams are in close proximity to each other (i.e., respective centers 33A and 33B of the focal zones are near each other, but do not coincide) within a target tissue region 34. For example, a distance D between respective centers 33A and 33B of the focal zones may be between 25% and 200% of the sum of a greatest diameter Q1 of focal zone 32A and a greatest diameter Q2 of focal zone 32B, such as between 50% and 100% of the sum of the diameters. (The greatest diameter of each focal zone is the diameter of the cross section of the focal zone that includes the center of the focal zone, as defined hereinbelow, and is perpendicular to the axis of the ultrasound beam.) For applications in which ultrasound beams 30A and 30B are parallel to each another, the axes of the beams are typically distance D from each other. Focal zones 32A and 32B may partially overlap (as shown in FIG. 1A), or may be non-overlapping (as shown in FIGS. 2-6). During a tissue destruction procedure, the axes and/or centers of the focal zones are typically maintained at distance D from each other for a time period of between 0.2 and 60 seconds (e.g., 0.2-5 seconds, or 5-60 seconds).

Because the wave propagation directions of the two focal zones are opposite to each another, beams 30A and 30B induce mechanical shear forces within the tissue region. The resulting shear forces tear and destroy at least a portion of the target tissue. The ultrasound beams may thus be considered to function as virtual scissors. Typically, signal generator 23 and a power amplifier that is connected thereto (power amplifier not shown) activate transducers 20A and 20B to generate the respective beams such that the beams arrive in the respective focal zones having the same phase.

As used in the present application, including in the claims, a focal point is a point at which the intensity of an ultrasound beam is maximal, and a focal zone is a volume surrounding the focal point, within which zone the intensity is reduced by up to −6 dB from the maximal intensity Typically, the focal zones produced by the focused ultrasound transducers have an elongated ellipsoidal shape, i.e., a solid of revolution generated by rotating an ellipse about its major axis, with the major axis coaxial with the axis of ultrasonic beams 30. For example, the focal zones may be generally cigar-shaped. The “center of the focal zone,” as used herein, including in the claims, is the location within the focal zone on the axis of the ultrasound beam at which the focal zone has a greatest cross-sectional area in a plane that is perpendicular to the axis of the ultrasound beam.

For applications in which the beams have a frequency of between 1 and 5 MHz, and target tissue region 34 comprises soft biological tissue, such as fat or muscle, each of focal zones 32 typically has a greatest diameter (perpendicular to the axis of the ultrasound beam) of between 0.5 and 2 mm, such as 1 mm, a greatest length (along the axis of the ultrasound beam) of between 3 and 15 times the diameter, such as between 8 and 10 times the diameter. For example, the diameter may be 1 mm and the length may be between 8 and 15 mm. For applications with such a frequency and for such tissue, the greatest cross-sectional area perpendicular to the axis of the ultrasound beam may be between 0.2 and 3 mm2, and the volume may be between 5 and 50 mm3.

If it is assumed that the intensity at the focal zone for each transducer 20 is I, a radiation pressure P is generated along the beam propagation direction having a value that is given by:

P=ηI/C (Equation 1)

where C is the speed of sound within the target tissue, and η is a factor which depends on the properties of the tissue, and typically has a value of between 1 and 2.

If it is assumed that the focal zone cross-sectional area is a, the resulting acoustic force is given by:

F=a×P (Equation 2)

The force direction is along the propagation direction of the waves. Because the two forces (generated by the respective two ultrasound beams) act in opposite directions and in close proximity; the forces apply shear to the target tissue located between the two focal zones. This generated shear may be considered analogous to the shear produced by a pair of scissors.

Controllable energy source 22 sets the intensity of the beams to be sufficient to cause damage to the target tissue, but typically without heating the tissue (e.g., not so intensive as to cause an increase in tissue temperature of at least 20° C.). For example, the tissue may have a specific heat of 4.18 J/(g*K).

Each of the transducers generates high intensity focused ultrasound (HIFU) beams, and comprises one or more focused transducers (e.g., piezoelectric elements), such as are described hereinbelow with reference to FIG. 2, or a phased array of transducer elements (e.g., piezoelectric elements), such as are described, hereinbelow with reference to FIGS. 3-7.

For some applications, signal generator 23 drives the transducers to continuously transmit ultrasonic waves (continuous wave mode). For other applications, the signal generator drives the transducers to transmit the waves in short pulses, e.g., each having a duration of between 1 and 1000 microseconds, e.g., 100 microseconds, with a duty cycle of between 5% and 30%, and a total application time of 1 to 60 seconds, e.g., 5 seconds. If an undesirable level of heating is generated using any of the parameters described herein, an option is to reduce the duty cycle to, for example, 5-10% or lower, or to reduce another one of the signal parameters.

For some applications, signal generator 23 drives the transducers to transmit at a frequency of between 1 and 10 MHz, such as between 2 and 5 MHz, e.g., 3 MHz, with approximately corresponding wavelengths of between 1.5 and 0.15 mm. As is known in the art, the dimensions of the focal zone depend on the wavelength; shorter wavelengths correspond to narrower focal zones. Typically, the length of the tear made in the tissue is approximately equal to one third of the length of the focal zones. Signal generator 23 may drive transducers 20A and 20B to transmit at either the same frequency or at different frequencies.

For some applications, signal generator 23 drives transducers 20A and 20B to generate sinusoidal waveforms. For other applications, the signal generator drives the transducers to generate arbitrary waveforms, such as chirp waveforms.

For some applications, as described hereinabove with reference to FIG. 1A, system 10 additionally comprises a mechanical or electromechanical system for spatially moving (position and/or orienting) the transducers with respect to housing 24. Such motion may be used to generally aim the transducers at target tissue region 34, and/or to precisely position the focal zones. For some applications, system 10 either automatically, or under manual control, positions the transducers in a plurality of directions during respective time periods, in order to destroy a plurality of tissue regions 34.

Reference is made to FIG. 2, which is a schematic illustration of a configuration of ultrasonic transducers 20A and 20B, in accordance with an application of the present invention. In this configuration, each of ultrasonic transducers 20A and 20B comprises one or more focused transducers, e.g., exactly one, which typically comprises a single transducer element 40 (e.g., a single piezoelectric element), which is configured to focus the generated ultrasound beam 30 in focal zone 32, as is known in the ultrasound art. Typically, system 10 is configured to focus each of the ultrasound beams such that the center of its focal zone is between 5 and 30 mm from the respective transducer. Such focus is typically fixed in this configuration.

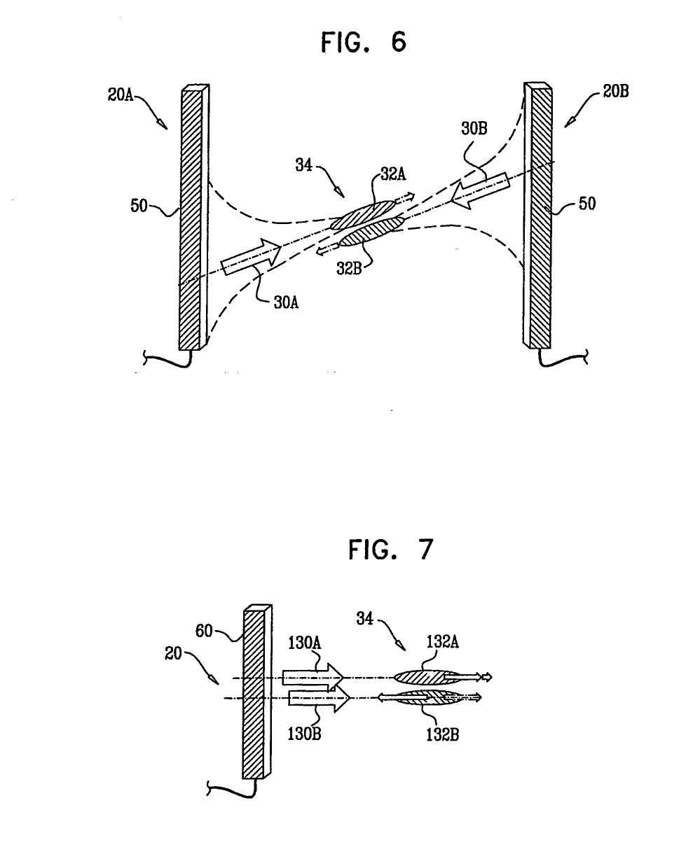

Reference is made to FIG. 3, which is a schematic illustration of another configuration of ultrasonic transducers 20A and 20B, in accordance with an application of the present invention. In this configuration, each of ultrasonic transducers 20A and 20B comprises a phased array 50 of transducer elements (e.g., piezoelectric elements), as is known in the ultrasound art. System 10 configures the beams such that the respective focal zones are in close proximity to each another by having energy source 22 activate the phased array to steer and focus the generated ultrasound beam in focal zones 32A and 32B. For some applications, a major axis of each focal zone defines an angle of 90 degrees with a surface of the phased array, as shown in FIG. 3; alternatively, the angle may be less than 90 degrees, as described hereinbelow with reference to FIG. 6. Although the phased arrays are shown as being linear in FIG. 3, the arrays may alternatively have another shape, such as an annular or other arbitrary shape.

For some applications, signal generator 23 is configured to steer the beams during the procedure so as to vary the directions and/or focal distances of the focal zones from the phased arrays during respective time periods. For some applications, as the focal distance of one of beams is increased, the focal distance of the other beam is correspondingly decreased, so that the two focal zones remain adjacent to each other. Such varying enables system 10 to destroy tissue in a plurality of tissue regions 34 without the need to physically move the transducers or applicator 12.

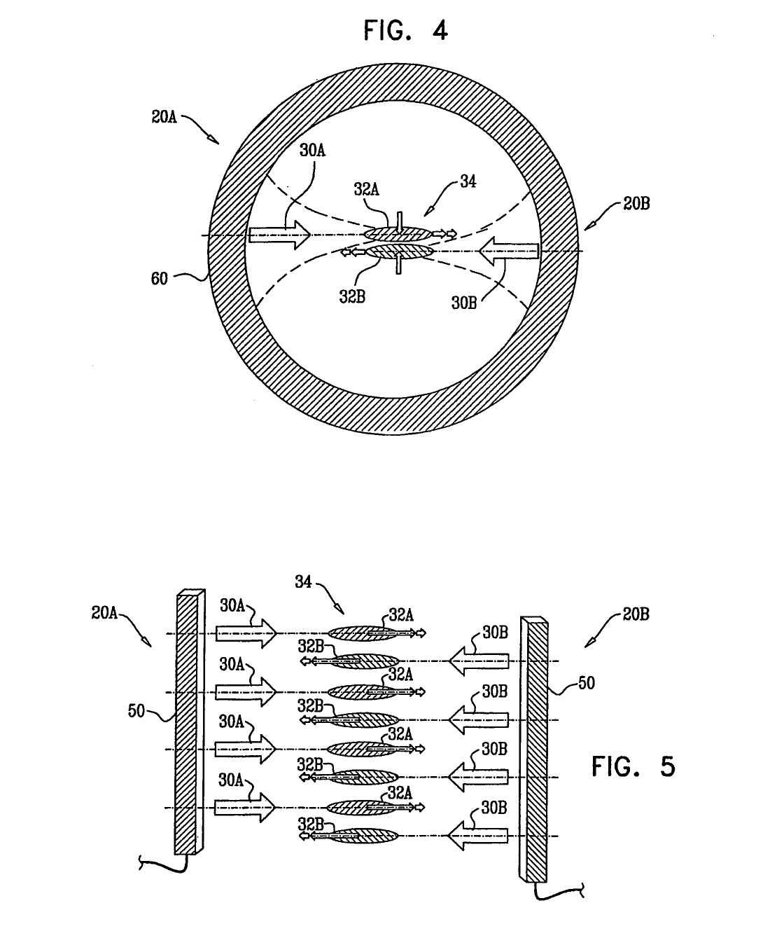

Reference is made to FIG. 4, which is a schematic illustration of yet another configuration of ultrasonic transducers 20A and 20B, in accordance with an application of the present invention. In this configuration, a phased array of transducer elements (e.g., piezoelectric elements) is arranged in a ring 60, and is configured to function as ultrasonic transducers 20A and 20B. Target tissue region 34 is pulled into the ring area by suction or another mechanical force. As a result, the ring surrounds the tissue circumferentially. Two subgroups of elements of ring 60 are configured to generate ultrasound beams 30A and 30B, respectively within a plane enclosed within the ring of the array, i.e., radially inward towards a central zone of the ring. (It is noted that this mode differs from a conventional annular array in which transmission is commonly perpendicular to the array plane.) The subgroups of elements may use conventional beam-forming techniques for generating the beams. Typically, the subgroups are non-overlapping, i.e., do not contain any common elements.

Signal generator 23 steers the location and orientation of focal zones 32A and 32B by manipulating the transmission amplitude and phase of the elements around the ring. This allows the tearing direction to be arbitrarily altered. Such control may be useful if one tissue direction is more rigid to shear stresses. In such a case, improved tissue destruction may be achieved by rotating the shear direction.

Reference is made to FIG. 5, which is a schematic illustration of still another configuration of ultrasonic transducers 20A and 20B, in accordance with an application of the present invention. In this configuration, each of ultrasonic transducers 20A and 20B comprises phased array 50 of transducer elements. Each of the arrays is configured to generate a plurality of adjacent, parallel ultrasound beams 30, either simultaneously or alternatingly, such that pairs of focal zones 32A and 32B thereof are close to each other but typically non-overlapping within target tissue region 34. Each of the pairs of beams induces mechanical shear forces within the tissue region. The resulting shear forces destroy the target tissue along a line comprising all of the focal zone pairs. For some applications, a major axis of each focal zone defines an angle of 90 degrees with a surface of the phased array, as shown in FIG. 3; alternatively, the angle may be less than 90 degrees, as described hereinbelow with reference to FIG. 6.

Reference is made to FIG. 6, which is a schematic illustration of a configuration of phased array 50, in accordance with an application of the present invention. In this application, signal generator 23 steers phased array 50 to change the orientation of focal zones 32A and 32B, and thus attain a tilted scissor-cutting effect of the beams. For example, the signal generator may set a major axis of each of the focal zones to define an angle of between 0 and 30 degrees with the normal to the surface of the phased array. This configuration enables the system to make an angular cut, and/or to make several cuts at the same position so as to more efficiently destroy tissue.

Reference is made to FIG. 7, which is a schematic illustration of a unidirectional transmission configuration of tissue destruction system 10, in accordance with an application of the present invention. In this application, system 10 comprises an ultrasonic transducer 20, which comprises a phased array 60 of transducer elements (e.g., piezoelectric elements), that is configured to be placed on one side of target tissue region 34, facing generally in the same direction, rather than on opposite sides as in the other configurations described hereinabove. Signal generator 23 is configured to drive array 60 to simultaneously generate at least one pair of two non-opposing ultrasound beams 60A and 60B, i.e., propagating in parallel in the same direction. Beams 60A and 60B have respective focal zones 132A and 132B in close proximity to each other within target tissue region 34, e.g., a distance between the respective centers of the focal zones is between 25% and 200% of the sum of the greatest diameters of focal zones 32A and 32B, such as between 50% and 100% of the sum of the diameters. The focal zones are typically maintained at this distance from each other for a time period of between 0.2 and 60 seconds (e.g., 0.2-5 seconds, or 5-60 seconds).

The signal generator configures the focal zones to have oppositely-signed pressures, i.e., it configures one of the focal zones to have a positive pressure pulse and the other of the focal zones to have a negative pressure pulse. For some applications, the signal generator repeatedly alternates the phases of the pulses so as to rapidly change the pressure gradient sign, e.g., at a pulse repetition frequency of between 100 and 5000 Hz, such as between 1 kHz and 3 kHz. The resulting pressure gradient formed between the two adjacent focal zones destroys the target tissue.

For some applications, control unit 18 (shown in FIG. 1) of system 10 performs a calibration procedure once or more during a procedure performed using system 10 (as described hereinabove with reference to any of the figures), in order to precisely control the locations of the focal zones within the target tissue and with respect to each other. For some applications, the control unit initially calibrates the ultrasound beams such that the respective focal zones coincide, such as by adjusting the timing or direction of one or both of the beams (either electrically, such as if the transducers comprise phased arrays, or mechanically or electromechanically). For example, the control unit may sense ultrasound intensity and ascertain that the focal zones maximally coincide if there is maximum cancellation or maximal through transmission between the two transducers. Alternatively, the system may generate the two beams at different frequencies, and sense the frequency of a third wave having a frequency that is equal to the difference between the frequencies of the two beams; the beams can be determined to have maximally coincided when the resultant frequency of the third wave is at its peak intensity. This calibration may be useful in particular if the tissue is heterogeneous, or if the mechanics of the applicator are not perfectly accurate. After this initial calibration, the system adjusts the location of at least one of (either exactly one of, or both of) the focal zones (either electrically or mechanically) such that the focal zones are in close proximity to each other, rather than coincide, as described hereinabove.

In an embodiment of the present invention, system 10 configures the ultrasound beams to comprise opposing focused shock waves (i.e., focused, high-intensity acoustic pulses) having focal zones located in close proximity to each other, using the techniques described hereinabove. For example, ultrasound transducers 20A and 20B may generate the shock waves using techniques used in lithotripsy, as is known in the art.

Although techniques of embodiments of the present invention have been described herein for tissue destruction, these techniques are also useful for tearing or otherwise destroying underlying structures in other materials (such as metal, silicon, plastic, or articles of manufacture).

For some applications, the phased arrays described hereinabove with reference to FIGS. 3, 5, 6, and 7 comprise planar phased arrays, instead of the linear arrays shown in, and described with reference to, these figures. The use of planar arrays enables steering of the ultrasound beams and focal zones in three dimensions, as is known in the art.

Although ultrasound transducers are sometimes described herein as comprising piezoelectric elements, non-piezoelectric elements, such as coil-activated membranes, electric spark systems, or laser-beam generators, may alternatively be used to generate acoustic waves in any of the embodiments described herein.

Techniques and apparatus described herein may be practiced in combination with techniques and apparatus described in one or more of the following patent applications, all of which are incorporated herein by reference:
? U.S. Provisional Patent Application 60/780,772 to Azhari et al., entitled, “A method and system for lypolysis and body contouring,” filed Mar. 9, 2006;
? U.S. Provisional Patent Application 60/809,577 to Azhari et al., entitled, “A device for ultrasound monitored tissue treatment,” filed May 30, 2006;
? U.S. Provisional Patent Application 60/860,635 to Azhari et al., entitled, “Cosmetic tissue treatment using ultrasound,” filed Nov. 22, 2006;
? U.S. Regular patent application Ser. No. 11/651,198 to Azhari et al., entitled, “A device for ultrasound monitored tissue treatment,” filed Jan. 8, 2007;
? U.S. Regular patent application Ser. No. 11/653,115 to Azhari et al., entitled, “A method and system for lipolysis and body contouring,” filed Jan. 12, 2007;
? PCT Patent Application Publication WO 07/102,161 to Azhari et al., entitled, “A device for ultrasound monitored tissue treatment,” filed Mar. 8, 2007;
? U.S. Provisional Patent Application 60/999,139 to Azhari et al., entitled, “Implosion techniques for ultrasound,” filed Oct. 15, 2007;
? U.S. Provisional Patent Application 61/096,419 to Azhari et al., entitled, “A device for ultrasound treatment and monitoring tissue treatment,” filed Sep. 12, 2008;
? PCT Patent Application Publication PCT/IL2008/001390 to Azhari et al., entitled, “Implosion techniques for ultrasound,” filed Oct. 22, 2008;
? U.S. Provisional Patent Application 61/096,419, entitled, “A device for ultrasound treatment and monitoring tissue treatment,” filed Sep. 12, 2008; and
? an international patent application filed on even date herewith claiming the benefit of the '419 application, entitled, “A device for ultrasound treatment and monitoring tissue treatment.”

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising:
at least first and second focused ultrasonic transducers, which are arranged facing each other; and
a controllable energy source, which is configured to activate the focused ultrasonic transducers to simultaneously generate respective first and second focused ultrasound beams having respective first and second focal zones located in close proximity to each other.

2. The apparatus according to claim 1, wherein the apparatus is configured to generate the respective beams such that a distance between respective centers of the respective focal zones is between 25% and 200% of the sum of a greatest diameter of the first focal zone and a greatest diameter of the second focal zone.

3.-4. (canceled)

5. The apparatus according to claim 1, wherein the apparatus is configured to generate the respective beams having respective opposing acoustic forces such that the beams together generate mechanical shear forces between the focal zones.

6. The apparatus according to claim 1, wherein the apparatus is configured to generate the respective beams such that the beams tear a material disposed between the focal zones.

7. The apparatus according to claim 1, wherein the apparatus is configured to generate the respective beams such that the beams do not increase a temperature of a material, having a specific heat of 4.18 J/(g*K), disposed between the focal zones, by more than 20° C.

8. The apparatus according to claim 7, wherein the apparatus is configured to generate the respective beams such that the beams do not cause substantial cavitation in the material.

9. The apparatus according to claim 1, further comprising a support structure, to which the focused ultrasonic transducers are coupled.

10. The apparatus according to claim 1, wherein the apparatus is configured to generate the respective beams such that the beams have parallel respective axes.

11. The apparatus according to claim 10, wherein the apparatus is configured to generate the respective beams such that a distance between the respective axes is between 25% and 200% of the sum of a greatest diameter of the first focal zone and a greatest diameter of the second focal zone.

12. (canceled)

13. The apparatus according to claim 1, wherein the focused ultrasonic transducers comprise respective phased arrays, and wherein the energy source is configured to activate the arrays to generate the respective focused ultrasound beams.

14. The apparatus according to claim 13, wherein the energy source is configured to activate each of the arrays to steer its respective focused ultrasound beam in a plurality of directions during respective time periods.

15. The apparatus according to claim 1, wherein the energy source is configured to mechanically steer the respective focused ultrasound beams.

16. The apparatus according to claim 1, wherein the focused ultrasonic transducers together comprise a phased array arranged in a ring, and wherein the energy source is configured to activate a first subgroup of the elements to generate the first focused ultrasound beam, and a second subgroup of the elements, different from the first subgroup, to generate the second focused ultrasound beam.

17. The apparatus according to claim 1, wherein the focused ultrasound beams are shock waves, and wherein the focused ultrasonic transducers are configured to simultaneously generate the shock waves having the respective first and second focal zones in close proximity to each other.

18. A method comprising:
identifying a target tissue region in a body of a subject; and
destroying at least a portion of the target tissue region by simultaneously generating, in opposing directions, at least first and second focused ultrasound beams having respective first and second focal zones in close proximity to each other within the target tissue region.

19. The method according to claim 18, wherein generating comprises generating the beams such that a distance between respective centers of the respective focal zones is between 25% and 200% of the sum of a greatest diameter of the first focal zone and a greatest diameter of the second focal zone.

20.-21. (canceled)

22. The method according to claim 18, wherein generating comprises generating the respective beams having respective opposing acoustic forces such that the beams together generate mechanical shear forces between the focal zones.

23. The method according to claim 18, wherein generating comprises generating the respective beams such that the beams tear tissue between the focal zones.

24.-25. (canceled)

26. The method according to claim 18, wherein generating comprises generating the respective beams such that the respective focused ultrasound beams have respective axes that are parallel to each other.

27. The method according to claim 26, wherein generating comprises generating the respective beams such that a distance between the respective axes is between 25% and 200% of the sum of a greatest diameter of the first focal zone and a greatest diameter of the second focal zone.

28. The method according to claim 18, wherein generating comprises:
positioning at least first and second focused ultrasonic transducers facing each other in a vicinity of the target tissue region, wherein the focused ultrasonic transducers include respective phased arrays; and
activating the respective phased arrays of the first and second focused ultrasonic transducers to simultaneously generate the first and second focused ultrasound beams, respectively.

29.-31. (canceled)

32. The method according to claim 18, wherein generating comprises steering the respective focused ultrasound beams.

33. (canceled)

34. The method according to claim 18, wherein generating the first and second focuses ultrasound beams comprises generating shock waves having the respective first and second focal zones in close proximity to each other.

35. Apparatus comprising:
at least first and second focused ultrasonic transducers, which are arranged facing in generally a same direction; and
a controllable energy source, which is configured to activate the focused ultrasonic transducers to simultaneously generate respective first and second focused ultrasound beams having respective first and second focal zones, which are located in close proximity to each other, and have oppositely-signed pressures.

36. The apparatus according to claim 35, wherein the apparatus is configured to generate the respective beams such that a distance between respective centers of the respective focal zones is between 25% and 200% of the sum of a greatest diameter of the first focal zone and a greatest diameter of the second focal zone.

37.-38. (canceled)

39. The apparatus according to claim 35, wherein the apparatus is configured to generate the respective beams having respective opposing acoustic forces such that the beams together generate mechanical shear forces between the focal zones.

40. The apparatus according to claim 35, wherein the apparatus is configured to generate the respective beams such that the beams tear a material disposed between the focal zones.

41. The apparatus according to claim 35, wherein the focused ultrasound beams are shock waves, and wherein the focused ultrasonic transducers are configured to simultaneously generate the shock waves having the respective first and second focal zones in close proximity to each other.

42. The apparatus according to claim 35, wherein the energy source is configured to activate the focused ultrasonic transducers to generate arbitrary waveforms.

43. A method comprising:
identifying a target tissue region in a body of a subject; and
destroying at least a portion of the target tissue region by simultaneously generating, in non-opposing directions, at least first and second focused ultrasound beams having respective first and second focal zones, which are in close proximity to each other within the target tissue region, and which have oppositely-signed pressures.

44. The method according to claim 43, wherein generating comprises generating the beams such that a distance between respective centers of the respective focal zones is between 25% and 200% of the sum of a greatest diameter of the first focal zone and a greatest diameter of the second focal zone.

45.-46. (canceled)

47. The method according to claim 43, wherein generating comprises generating the respective beams having respective opposing acoustic forces such that the beams together generate mechanical shear forces between the focal zones.

48. The method according to claim 43, wherein generating comprises generating the respective beams such that the beams tear tissue between the focal zones.

49. The method according to claim 43, wherein generating the first and second focuses ultrasound beams comprises generating shock waves having the respective first and second focal zones in close proximity to each other.

50. The method according to claim 43, wherein generating comprises generating the beams having arbitrary waveforms.

51. The apparatus according to claim 1, wherein the apparatus is configured to perform a calibration procedure, in which the apparatus initially generates the first and second ultrasound beams such that the respective focal zones coincide, and thereafter adjusts a location of at least one of the focal zones such that the focal zones are in close proximity to each other, rather than coincide.

52. The method according to claim 18, wherein generating comprises performing a calibration procedure, which comprises:
initially generating the first and second ultrasound beams such that the respective focal zones coincide; and
thereafter adjusting a location of at least one of the focal zones such that the focal zones are in close proximity to each other, rather than coincide.

53. The apparatus according to claim 35, wherein the apparatus is configured to perform a calibration procedure, in which the apparatus initially generates the first and second ultrasound beams such that the respective focal zones coincide, and thereafter adjusts a location of at least one of the focal zones such that the focal zones are in close proximity to each other, rather than coincide.

54. The method according to claim 43, wherein generating comprises performing a calibration procedure, which comprises:
initially generating the first and second ultrasound beams such that the respective focal zones coincide; and
thereafter adjusting a location of at least one of the focal zones such that the focal zones are in close proximity to each other, rather than coincide.
